(19) United States
(12) Patent Application Publication
(10) Pub. No.: US 2005/0069523 A1
(43) Pub. Date: Mar. 31, 2005

(54) Recombinant adenoviruses encoding glial cell neurotrophic factor (GDNF)

(75) Inventors: Philippe Horellou, Paris (FR); Jacques Mallet, Paris (FR); Michel Perricaudet, Ecrosnes (FR); Frederic Revah, Paris (FR); Emmanuelle Vigne, Ivry Sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: RHONE-POULENC RORER S.A.

(21) Appl. No.: 10/614,794

(22) Filed: Jul. 9, 2003

Related U.S. Application Data
(63) Continuation of application No. 08/904,872, filed on Aug. 1, 1997, now abandoned.
Application No. 08/904,872 is a continuation-in-part of application No. 08/716,326, filed on Oct. 4, 1996, now Pat. No. 6,245,330.

(30) Foreign Application Priority Data
Mar. 25, 1994 (FR) FR94/03542

(51) Int. Cl. A61K 48/00; C12N 7/00; C12N 15/861

(52) U.S. Cl. 424/93.2; 435/456; 435/235.1

(57) ABSTRACT

Recombinant adenoviruses comprising a heterologous DNA sequence coding for glial-derived neurotrophic factor (GDNF), preparation thereof, and use thereof for treating and/or preventing degenerative neurological diseases.

The present invention relates to recombinant adenoviruses which contain a DNA sequence encoding the glial cell-derived neurotrophic factor. The invention also relates to the preparation of these vectors, to the pharmaceutical compositions which contain them, and to their therapeutic use, especially in gene therapy, for treating and/or preventing neurodegenerative diseases.

The increase in the length of life in Western countries is accompanied by a steady growth in neurodegenerative diseases such as Alzheimer's disease, Parkinson's disease, Huntington's chorea, amyotrophic lateral sclerosis, etc. Thus, Parkinson's disease, for example, affects 4% of people above the age of 65, and Alzheimer's disease affects 10% of those above the age of 70 and 30% of those above the age of 80. Generally speaking, all these diseases result from a progressive loss of neuronal cells in the central nervous system, or even within very localized structures, as in the case of Parkinson's disease.

During recent years, numerous research programmes have been developed in order to understand the mechanisms of this degeneration associated with ageing, with a view to developing means for treating it, and also for preventing it, by gene therapy.

Since the neurodegenerative diseases are an expression of the progressive death of the neuronal cells, stimulation of the production of the growth factors involved in the development of these neuronal cells has in fact appeared to be a possible route for preventing and/or opposing this degeneration.

The object of the present invention is, in particular, to propose vectors which make it possible directly to promote the survival of the neuronal cells which are involved in these pathologies by means of expressing, in an efficient and localized manner, certain trophic factors.

The trophic factors are a class of molecules which possess properties of stimulating axonal growth or the survival of the nerve cells. The first factor possessing neurotrophic properties, NGF (“Nerve Growth Factor”), was characterized some 40 years ago (for review, see Levi-Montalcini and Angelleti, Physiol. Rev. 48 (1968) 534). Other neurotrophic factors, in particular the glial cell-derived neurotrophic factor (GDNF) (L.-F. Lin, D. Doherty, J. Lile, S. Besktesh, F. Collins, Science, 260, 1130-1132 (1993)) have only been identified recently. GDNF is a protein of 134 amino acids with a molecular weight of 16 kD. Its essential function is the in-vitro promotion of the survival of dopaminergic neurones.

The present invention is particularly advantageous for administering GDNF in the form of a therapeutic agent.

More precisely, the present invention is directed towards developing vectors which are particularly effective in delivering, in vivo and in a localized manner, therapeutically active quantities of the specific gene encoding GDNF in the nervous system.

In application No. PCT/EP93/02519, which is pending concomitantly, it was demonstrated that it was possible to use the adenoviruses as vectors for transferring a foreign gene in vivo into the nervous system and expressing the corresponding protein.

More specifically, the present invention relates to specially adapted and efficient novel constructs for transferring glial cell-derived neurotrophic factor (GDNF).

More precisely, it relates to a recombinant adenovirus which encompasses a DNA sequence encoding GDNF or one of its derivatives, to its preparation, and to its use for treating and/or preventing neurodegenerative diseases.

Thus, the Applicant has clearly demonstrated that it is possible to construct recombinant adenoviruses which contain a sequence encoding GDNF, and to administer these recombinant adenoviruses in vivo, and that this administration permits stable and localized expression of therapeutically active quantities of GDNF in vivo, in particular in the nervous system and without any cytopathic effect.

An initial subject of the invention is thus a defective recombinant adenovirus which encompasses at least one DNA sequence encoding all, or an active part, of the glial cell-derived neurotrophic factor (GDNF) or one of its derivatives.

The glial cell-derived neurotrophic factor (GDNF) which is produced within the scope of the present invention can either be human GDNF or an animal GDNF.

The cDNA sequences encoding human GDNF and rat GDNF have been cloned and sequenced (L.-F. Lin, D. Doherty, J. Lile, S. Besktesh, F. Collins, Science, 260, 1130-1132 (1993)).

The DNA sequence which encodes GDNF and which is used within the scope of the present invention can be a cDNA, a genomic DNA (gDNA), or a hybrid construct consisting, for example, of a cDNA in which one or more introns could be inserted. The sequence may also consist of synthetic or semisynthetic sequences. Particularly advantageously, the sequence of the present invention encodes GDNF which is preceded by the native pro region (pro GDNF).

Particularly advantageously, a cDNA or a gDNA is employed. According to a preferred embodiment of the invention, the sequence is a gDNA sequence encoding GDNF. Use of this latter sequence can make it possible to achieve improved expression in human cells.

Naturally, prior to its incorporation into an adenovirus vector according to the invention, the DNA sequence is advantageously modified, for example by site-directed mutagenesis, especially in order to insert appropriate restriction sites. Thus, the sequences described in the prior art are not constructed so that they can be used in accordance with the invention, and preliminary adaptations may prove to be necessary in order to obtain a substantial level of expression.

Within the meaning of the present invention, a derivative of GDNF is understood to mean any sequence which is obtained by modification and which encodes a product which retains at least one of the biological properties of GDNF (trophic effect and/or differentiating effect). Modification should be understood to mean any mutation, substitution, deletion, addition or modification of a genetic and/or chemical nature. These modifications can be effected by techniques known to the person skilled in the art (see general molecular biological techniques below). The derivatives within the meaning of the invention can also be obtained by hybridization from nucleic acid libraries, using the native sequence or a fragment thereof as the probe.

These derivatives are, in particular, molecules which have a greater affinity for their sites of attachment, sequences which permit improved expression in vivo, molecules which are more resistant to proteases, and molecules which have greater therapeutic efficacy or less pronounced secondary effects, or, perhaps, novel biological properties.

The preferred derivatives which may most particularly be cited are natural variants, molecules in which one or more residues have been replaced, derivatives which have been obtained by deleting regions which are not involved, or only involved to a limited extent, in the interaction with the binding sites under consideration, or which express an undesirable activity, and derivatives which include residues which are additional to those in the native sequence, such as, for example, a secretory signal and/or a junction peptide.

According to one preferred embodiment of the invention, the DNA sequence encoding GDNF or one of its derivatives also includes a secretory signal which makes it possible to direct the synthesized GDNF into the secretory paths of the infected cells. According to one preferred embodiment, the DNA sequence contains a secretory sequence in the 5′ position and in reading frame with the sequence encoding the GDNF. In this way, the synthesized GDNF is advantageously released into the extracellular compartments and can in this way activate its receptors. The secretory signal is advantageously the native secretory signal of the GDNF (referred to below by the term “pre”). However, the secretory signal can also be a secretory signal which is heterologous or even artificial. Advantageously, the DNA sequence encodes pre-GDNF or, more particularly, human pre-GDNF.

Advantageously, the sequence encoding GDNF is placed under the control of signals which allow the GDNF to be expressed in nerve cells. Preferably, these signals are heterologous expression signals, that is signals which are different from those which are naturally responsible for expressing GDNF. They may, in particular, be sequences which are responsible for expressing other proteins, or synthetic sequences. In particular, they can be promoter sequences from eucaryotic or viral genes. For example, they can be promoter sequences derived from the genome of the cell which it is wished to infect. Similarly, they can be promoter sequences derived from the genome of a virus, including the adenovirus being used. Examples of promoters which may be cited in this regard are E1A, MLP, CMV, RSV LTR, etc. Furthermore, these expression sequences can be modified by adding activation sequences or regulatory sequences, or sequences which allow tissue-specific expression. Thus, it can be of particular interest to use expression signals which are active specifically, or in the main, in nerve cells, such that the DNA sequence is only expressed, and only produces its effect, when the virus has actually infected a nerve cell. Examples of promoters which may be cited in this respect are those of the neurone-specific enolase, of GFAP, etc.

In a first specific embodiment, the invention relates to a defective recombinant adenovirus which includes a cDNA sequence encoding human pre-GDNF under the control of the RSV LTR promoter.

In a second specific embodiment, the invention relates to a defective recombinant adenovirus which includes a gDNA sequence encoding human pre-GDNF under the control of the RSV LTR promoter.

Thus, the Applicant has demonstrated that the LTR promoter of the Rous sarcoma virus (RSV) enabled GDNF to be expressed over a long period and at a substantial level in the cells of the nervous system, in particular of the central nervous system.

Still within a preferred embodiment, the invention relates to a defective recombinant adenovirus which includes a DNA sequence encoding the whole, or an active part, of human GDNF, or of a derivative thereof, under the control of a promoter which enables most expression to take place in the nervous system.

A particularly preferred embodiment of the present invention is a defective recombinant adenovirus which includes the ITR sequences, a sequence allowing encapsidation, and a DNA sequence encoding glial cell-derived human neurotrophic factor (hGDNF), or a derivative thereof, under the control of a promoter allowing most of the expression to take place in the nervous system, and in which the E1 gene, and at least one of the genes E2, E4 and L1-L5 is non-functional.

Defective adenoviruses according to the invention are adenoviruses which are incapable of replicating autonomously in the target cell. In general, the genome of the defective adenoviruses used within the scope of the present invention therefore lacks at least those sequences which are necessary for the said virus to replicate in the infected cell. These regions may be removed (in whole or in part), or rendered non-functional, or replaced by different sequences, in particular by the DNA sequence encoding GDNF.

The defective virus of the invention preferably retains those sequences of its genome which are necessary for encapsidating the viral particles. Still more preferably, as indicated above, the genome of the defective recombinant virus according to the invention includes the ITR sequences, a sequence allowing encapsidation, the non-functional E1 gene, and a non-functional version of at least one of the genes E2, E4 and L1-L5.

Different serotypes of adenovirus exist, whose structures and properties vary to some degree. Of these serotypes, preference is given to using the type 2 or type 5 human adenoviruses (Ad 2 or Ad 5) or the adenoviruses of animal origin (see application FR 93 05954) within the scope of the present invention. Adenoviruses of animal origin which can be used within the scope of the present invention and which may be mentioned are the adenoviruses of canine, bovine, murine (example: Mav1, Beard et al., Virology 75 (1990) 81), ovine, porcine, avian and also simian (example: SAV) origin. The adenovirus of animal origin is preferably a canine adenovirus, more preferably a CAV2 adenovirus [Manhattan strain or A26/61 (ATCC VR-800) for example]. Adenoviruses of human or canine origin, or a mixture of these, are preferably employed within the scope of the invention.

The defective recombinant adenoviruses according to the invention can be prepared by any technique known to the person skilled in the art (Levrero et al., Gene 101 (1991) 195, EP 185 573; Graham, EMBO J. 3 (1984) 2917). In particular, they can be prepared by homologous recombination between an adenovirus and a plasmid which carries, inter alia, the DNA sequence encoding GDNF. The homologous recombination takes place after cotransfection of the said adenovirus and plasmid into an appropriate cell line. The cell line which is employed should preferably (i) be transformable by the said elements, and (ii) contain the sequences which are able to complement the defective adenovirus genome part, preferably in an integrated form in order to avoid the risk of recombination. As an example of a cell line, mention may be made of the human embryonic kidney cell line 293 (Graham et al., J. Gen. Virol. 36 (1977) 59) which contains, in particular, integrated into its genome, the left-hand part of the genome of an Ad5 adenovirus (12%). Strategies for constructing vectors derived from adenoviruses have also been described in applications Nos. FR 93 05954 and FR 93 08596, which are incorporated herein by reference.

Afterwards, the adenoviruses which have multiplied are recovered and purified using conventional molecular biological techniques, as illustrated in the examples.

The properties of the vectors of the invention which are particularly advantageous ensue, in particular, from the construct employed (defective adenovirus, in which certain viral regions are deleted), from the promoter which is employed for expressing the sequence encoding GDNF (preferably a viral or tissue-specific promoter), and from the methods of administering the said vector, resulting in an expression of GDNF which is efficient and which takes place in the appropriate tissues. The present invention thus provides viral vectors which can be employed directly in gene therapy, and which are particularly suitable and efficient for directing expression of GDNF in vivo. The present invention thus offers a novel approach which is particularly advantageous for treating and/or preventing neurodegenerative diseases.

The present invention also relates to any employment of an adenovirus such as described above for preparing a pharmaceutical composition which is intended for treating and/or preventing neurodegenerative diseases.

More especially, it relates to any employment of these adenoviruses for preparing a pharmaceutical composition which is intended for treating and/or preventing Parkinson's disease, Alzheimer's disease, amyotrophic lateral sclerosis (ALS), Huntington's disease, epilepsy and vascular dementia.

The present invention also relates to a pharmaceutical composition which includes one or more defective recombinant adenoviruses such as those previously described. These pharmaceutical compositions can be formulated with a view to administering them by the topical, oral, parenteral, intranasal, intravenous, intramuscular, subcutaneous, intraocular or transdermal, route, inter alia. Preferably, the pharmaceutical compositions of the invention contain an excipient which is pharmaceutically acceptable for an injectable formulation, in particular for injection directly into the nervous system of the patient. These injectable formulations can, in particular, be sterile, isotonic solutions, or dry, in particular lyophilized, compositions which, by means of sterile water or physiological saline, as the case may be, being added to them, enable injectable solutions to be constituted. Direct injection into the nervous system of the patient is advantageous since it enables the therapeutic effect to be concentrated at the level of the affected tissues. Direct injection into the central nervous system of the patient is advantageously effected using a stereotactic injection apparatus. The reason for this is that use of such an apparatus renders it possible to target the injection site with a high degree of precision.

In this respect, the invention also relates to a method for treating neurodegenerative diseases which comprises administering a recombinant adenovirus such as defined above to a patient. More especially, the invention relates to a method for treating neurodegenerative diseases which comprises stereotactically administering a recombinant adenovirus such as defined above.

The doses of defective recombinant adenovirus which are employed for the injection can be adjusted depending on different parameters, in particular depending on the mode of administration employed, on the pathology concerned, and also on the sought-after duration of the treatment. Generally, the recombinant adenoviruses according to the invention are formulated and administered in the form of doses consisting of between 10⁴ and 10¹⁴ pfu/ml, preferably from 10⁶ to 10¹⁰ pfu/ml. The term pfu (“plaque-forming unit”) represents the infective power of a virus solution, and is determined by infecting an appropriate cell culture and then measuring, in general after 48 hours, the number of plaques of infected cells. The techniques for determining the pfu titre of a viral solution are well documented in the literature.

The invention also relates to any mammalian cell which is infected with one or more defective recombinant adenoviruses such as described above. More especially, the invention relates to any population of human cells which is infected with these adenoviruses. These cells can, in particular, be fibroblasts, myoblasts, hepatocytes, keratinocytes, endothelial cells, glial cells, etc.

The cells according to the invention can be derived from primary cultures. These cells can be removed by any technique known to the person skilled in the art and then cultured under conditions which allow them to proliferate. As regards fibroblasts, more especially, these cells can readily be obtained from biopsies, for example using the technique described by Ham [Methods Cell. Biol. 21a (1980) 255]. These cells can be employed directly for infection with the adenoviruses, or be preserved, for example by freezing, in order to establish autologous banks for subsequent use. These cells according to the invention can also be secondary cultures which are obtained, for example, from pre-established banks.

The cells in culture are then infected with recombinant adenoviruses in order to confer on the cells the capacity to produce GDNF. The infection is carried out in vitro using techniques known to the person skilled in the art. In particular, the person skilled in the art can adjust the multiplicity of infection and, where appropriate, the number of cycles of infection which is carried out, in accordance with the type of cells employed and with the number of virus copies per cell which is required. Naturally, these steps have to be performed under appropriate conditions of sterility since the cells are destined for in-vivo administration. The doses of recombinant adenovirus which are employed for infecting the cells can be adjusted by the person skilled in the art in accordance with the sought-after objective. The conditions described above for administration in vivo can be applied to infection in vitro.

The invention also relates to an implant comprising mammalian cells which are infected with one or more defective recombinant adenoviruses as described above, and an extracellular matrix. Preferably, the implants according to the invention comprise from 10⁵ to 10¹⁰ cells. More preferably, they comprise from 10⁶ to 10⁸ cells.

More especially, the extracellular matrix in the implants of the invention comprises a gel-forming compound and, where appropriate, a support for anchoring the cells.

Different types of gel-forming agents can be employed for preparing implants according to the invention. The gel-forming agents are used in order to enclose the cells in a matrix having a gel constitution, and, if the need arises, in order to facilitate anchorage of the cells on the support. Various cell adhesion agents can, therefore, be used as gel-forming agents, such as, in particular, collagen, gelatin, glycosaminoglycans, fibronectin, lectins, etc. Collagen is preferably used within the scope of the present invention. This collagen can be of human, bovine or murine origin. More preferably, type I collagen is used.

As indicated above, the compositions according to the invention advantageously comprise a support for anchoring the cells. The term anchoring denotes any form of biological and/or chemical and/or physical interaction leading to adhesion and/or attachment of the cells to the support. Moreover, the cells can cover the support which is used and/or penetrate into the interior of this support. Within the scope of the invention, preference is given to using a non-toxic and/or biocompatible solid support. In particular, use may be made of polytetrafluoroethylene (PTFE) fibres or of a support of biological origin.

The implants according to the invention can be implanted at different sites in the organism. In particular, implantation can be effected at the level of the peritoneal cavity, in subcutaneous tissue (suprapubic region, iliac or inguinal fossae, etc.), in an organ, a muscle, a tumour, the central nervous system, and also under a cornification. The implants according to the invention are particularly advantageous in that they make it possible to control the release of the therapeutic product within the organism: this release is initially determined by the multiplicity of infection and by the number of implanted cells. After that, the release can be controlled by the shrinkage of the implant, which definitively stops the treatment, or by using regulatable expression systems which enable expression of the therapeutic genes to be induced or repressed.

The present invention thus offers a very efficient means for treating and/or preventing neurodegenerative diseases. It is quite particularly adapted for treating Alzheimer's, Parkinson's and Huntington's diseases, and for treating ALS. Furthermore, the adenoviral vectors according to the invention display important advantages which are linked, in particular, to their very high efficiency in infecting nerve cells, thereby making it possible to achieve infections using low volumes of viral suspension. In addition, infection with the adenoviruses of the invention is localized to a high degree to the site of injection thereby avoiding the risk of any diffusion into adjacent cerebral structures.

Furthermore, this treatment can be used just as easily for humans as for any animal such as sheep, cattle, domestic animals (dogs, cats, etc.), horses, fish, etc.

The present invention will be described in more detail using the following examples, which must be regarded as illustrating the invention and not limiting it.

### BRIEF DESCRIPTION OF THE DRAWINGS

FIG. 1: Depiction of the vector pLTR IX-GDNF.

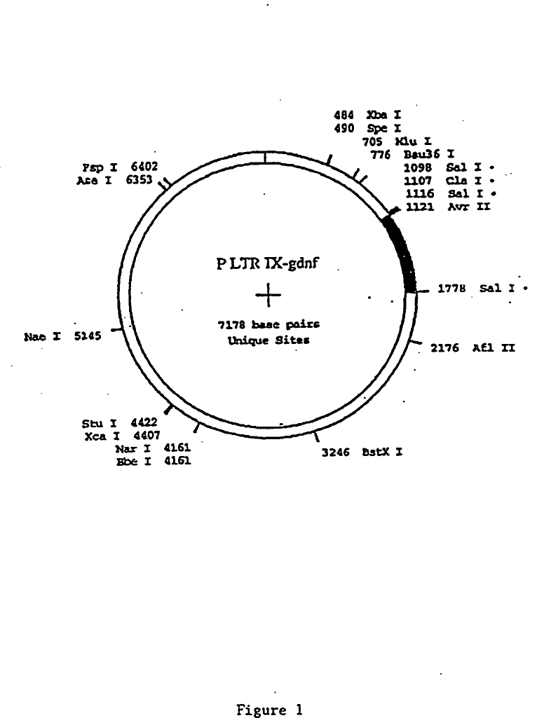

FIG. 2. Analysis of adenoviral transgene expression using βGal-immunohistochemistry. Pictures of 14 μm-thick coronal sections through the caudate putamen (A) and substantia nigra (B) showing βGal-expressing cells four weeks after intrastriatal injection of Ad-βGal. Anti E. coli-βGal antibodies were used to distinguish the transgenic from the endogenous βGal activity. In the striatum, βGal (+) cells are found along the needle tract (indicated by arrows in A), and up to 2 mm from the site of injection. Numerous infected cells can be observed in the substantia nigra compacta (B) following retrograde transport of viral particles delivered in the caudate putamen. Bar corresponds to 200 mm.

FIG. 3. Survival of dopaminergic-neurons in the substantia nigra of 6-OHDA lesioned rats. The animals received intrastriatal Ad injections followed by 6-OHDA 6 days later. Three weeks after 6-OHDA injection, animals were sacrificed for tyrosine hydroxylase-immunohistochemistry. The number of tyrosine hydroxylase (+) cell bodies present in the substantia nigra at the coordinates AP −4.8, −5.3 and −5.8 mm from bregma (3-4 sections per region of each animal) was determined. The values reported are means for 6-11 rats per group±SEM and are expressed as percentages of tyrosine hydroxylase (+) cell counts in the contralateral non-lesioned substantia nigra. The survival of dopaminergic-neurons is significantly higher in animals injected with Ad-GDNF (□) than with Ad-βGal (▪) or than in animals that received 6-OHDA alone (O). **, P<0.01 versus 6-OHDA alone; φ, P<0.01 and ‡, p<0.001 vs Ad-βGal.

FIG. 4. Histological analysis of substantia nigra dopaminergic-neurons of treated rats. Representative pictures of 14 μm-thick coronal sections through the substantia nigra processed for tyrosine hydroxylase-immunohistochemistry are shown. (A) section contralateral to the lesion, (B and C) sections ipsilateral to the lesion of animals injected with Ad-βGal (B), or Ad-GDNF (C). The aspect of the ipsilateral substantia nigra from rats that received only 6-OHDA is comparable to those of rats that received 6-OHDA+Ad-βGal (see B). Scale bar corresponds to 100 μm. The number of tyrosine hydroxylase (+) cell bodies and density of tyrosine hydroxylase-stained fibers were both higher in rats which received Ad-GDNF than Ad-βGal (C versus B).

FIG. 5. Effect of Ad-GDNF on amphetamine-induced rotational behavior in 6-OHDA-lesioned rats. Ad-GDNF (n=7) or Ad-βGal (n=8) were delivered into the left striatum of animals by stereotaxic injection. Six days thereafter, 20 μg of 6-OHDA-hydrochloride was injected into the left striatum of all two groups of animal. A third group of animals received no pre-injection before 6-OHDA lesion (6-OHDA only, n=10). The ability of the different treatments to counteract the neurotoxin action was assessed by following asymmetric rotational behavior induced by amphetamine administration 1, 2 and 3 weeks after 6-OHDA injection. The values reported are means±SEM (bars) of net ipsilateral turns over 90 min (turns contralateral to the lesion subtracted). *, p<0.05; ***, p<0.001 and ns: not significant vs 6-OHDA alone. #, p<0.05 and φ, p<0.01 vs Ad-βGal.

### GENERAL MOLECULAR BIOLOGICAL TECHNIQUES

The standard methods employed in molecular biology such as preparative extractions of plasmid DNA, centrifugation of plasmid DNA in a caesium chloride gradient, electrophoresis on agarose or acrylamide gels, purification of DNA fragments by electroelution, extraction of proteins with phenol or with phenol/chloroform, precipitation of DNA in a saline medium using ethanol or isopropanol, transformation into Escherichia coli, etc., are well known to the person skilled in the art and are widely described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Protocols in Molecular Biology”, John Wiley & Sons, New York, 1987].

The plasmids such as pBR322 and pUC, and the phages of the M13 series were obtained commercially (Bethesda Research Laboratories).

For the ligations, the DNA fragments can be separated according to their size by electrophoresis in agarose or acrylamide gels, extracted with phenol or with a phenol/chloroform mixture, precipitated by ethanol and then incubated in the presence of T4 phage DNA ligase (Biolabs) in accordance with the supplier's instructions.

The protruding 5′ ends can be filled in using the Klenow fragment of E. coli DNA polymerase I (Biolabs) in accordance with the supplier's specifications. The protruding 3′ ends are destroyed in the presence of T4 phage DNA polymerase (Biolabs), which is employed in accordance with the manufacturer's instructions. The protruding 5′ ends are destroyed by careful treatment with S1 nuclease.

In vitro site-directed mutagenesis using synthetic oligodeoxynucleotides can be performed using the method developed by Taylor et al. [Nucleic Acids Res. 13 (1985) 8749-8764] and employing the kit distributed by Amersham.

Enzymic amplification of DNA fragments by the technique termed PCR [polymerase-catalysed chain reaction, Saiki R. K. et al., Science 230 (1985) 1350-1354; Mullis K. B. et Faloona F. A., Meth. Enzym. 155 (1987) 335-350] can be performed using a “DNA thermal cycler” (Perkin Elmer Cetus) in accordance with the manufacturer's specifications.

The nucleotide sequences can be verified by means of the method developed by Sanger et al. [Proc. Natl. Acad. Sci. USA, 74 (1977) 5463-5467] using the kit distributed by Amersham.

### EXAMPLES

### Example 1

### Construction of the Vector pLTR IX-GDNF

This example describes the construction of the vector pLTR IX-GDNF, which contains the sequence encoding rat pre-GDNF under the control of the RSV virus LTR, as well as adenovirus sequences which permit in-vivo recombination.

Cloning of a cDNA encoding rat pre-GDNF. The cloning is effected by means of the PCR technique, which makes use of rat glial cell cDNA which is obtained by reverse transcription of RNA derived from these cells, employing the following oligonucleotides as templates:

| | |
|---|---|
| 5′ Oligonucleotide: | |
| CCGTCGACCTAGGCCACCATGAAGTTATGGGATGTC | (SEQ ID NO: 1) |
| 3′ Oligonucleotide: | |
| CCGTCGACATGCATGAGCTCAGATACATCCACACC | (SEQ ID NO: 2) |

After the fragments obtained by the PCR technique had been subjected to gel purification and cut with the restriction enzyme SalI, they were inserted into a Bluescript (Stratagene) plasmid in the SalI site. A polyadenylation sequence derived from SV40 had previously been introduced into the XhoI site of the same plasmid. This plasmid is termed SK-GDNF-PolyA.

The vector pLTRIX-GDNF was obtained by introducing an insert, obtained by cutting SK-GDNF-PolyA with ClaI and KpnI (KpnI ends rendered blunt), between the ClaI and EcoRV sites of the plasmid pLTRIX (Stratford, Perricaudet et al., J; Clin. Invest. 90(1992) p 626).

### Example 2

### Construction of Recombinant Adenoviruses Containing a Sequence Encoding GDNF

The vector pLTR IX-GDNF was linearized and cotransfected together with a defective adenoviral vector into helper cells (cell line 293) supplying the functions encoded by the adenovirus E1 (E1A and E1B) regions in trans.

More precisely, the adenovirus Ad-GDNF was obtained by means of in-vivo homologous recombination between the mutant adenovirus Ad-d11324 (Thimmappaya et al., Cell 31 (1982) 543) and vector pLTR IX-GDNF, in accordance with the following protocol: plasmid pLTR IX-GDNF and adenovirus Ad-d11324, linearized with the enzyme ClaI, were cotransfected into cell line 293 in the presence of calcium phosphate in order to enable homologous recombination to take place. The recombinant adenoviruses which were thereby generated were selected by plaque purification. Following isolation, the DNA of the recombinant adenovirus was amplified in cell line 293, resulting in a culture supernatent being obtained which contains non-purified defective recombinant adenovirus having a titre of approximately 10¹⁰ pfu/ml.

The virus particles are subsequently purified by gradient centrifugation.

### Example 3

### In-vivo Transfer of th GDNF Gene by means of a Recombinant Adenovirus into Rats having a Lesion in the Nigrostriatal Tract

This example describes the in-vivo transfer of the GDNF gene using an adenoviral vector according to the invention. It demonstrates, using an animal model of the nigrostriatal tract lesion, that the vectors of the invention render it possible to induce expression of therapeutic quantities of GDNF in vivo.

The nigrostriatal tract of rats which had previously been anaesthetized was damaged at the level of the median mesencephalic tract (MFB) by injecting the toxin 6-hydroxydopamine (60H-DA). This chemical lesion induced by injection was unilateral, in accordance with the following stereotactic coordinates: AP: 0 and −1; ML: +1.6; V: −8.6 and −9 (the AP and ML coordinates are determined in relation to the bregma, and the V coordinate in relation to the dura mater). The line of incision is fixed at the level +5 mm.

Immediately after the lesion had been made, the recombinant GDNF adenovirus was injected into the substantia nigra and the striatum on the side of the lesion. More especially, the adenovirus which is injected is the Ad-GDNF adenovirus, which was previously prepared and which was used in purified form (3.5×10⁶ pfu/μl) in a phosphate-buffered saline (PBS) solution.

The injections were carried out using a canula (280 μm external diameter) which was connected to a pump. The speed of injection is fixed at 0.5 μl/min, after which the canula remains in place for a further 4 minutes before being removed. The volumes injected into the striatum and the substantia nigra are 2×3 μl and 2 μl, respectively. The concentration of adenovirus which is injected is 3.5×10⁶ pfu/μl.

The following stereotactic coordinates are used for injection into the substantia nigra: AP=−5.8; ML=+2; V=−7.5 (the AP and ML coordinates are determined in relation to the bregma and the V coordinate in relation to the dura mater).

The following stereotactic coordinates are used for the injections into the striatum: AP=+0.5 and −0.5; ML=3; V=−5.5 (the AP and ML coordinates are determined in relation to the bregma, and the V coordinate in relation to the dura mater).

The therapeutic effects of administering the adenovirus according to the invention were demonstrated by three types of analysis: histological and immunohistochemical analysis, quantitative analysis and behavioural analysis.

Histological and Immunohistochemical Analysis

The chemical lesion in the nigrostriatal tract induces neuronal loss in the substantia nigra as well as dopaminergic denervation in the striatum (changes which are revealed in immunohistology by means of using an anti-tyrosine hydroxylase, TH, antibody).

Histological analysis of the injected brains is carried out three weeks after injecting the Ad-GDNF adenovirus intracerebrally under the conditions described in Example 6. Serial coronal sections of 30 μm in thickness are taken from the substantia nigra and the striatum. Sections spaced at intervals of 180 μm (1 section in 6) are stained with cresyl violet (in order to assess neuronal density) and immunolabelled with an anti-tyrosine hydroxylase (TH) antibody (in order to detect the dopaminergic neurones in the substantia nigra and their innervation in the striatum).

Quantitative Analysis

The number of dopaminergic neurones (TH-positive) in the substantia nigra is the parameter for evaluating the effects of the Ad-GDNF adenovirus. Counting is carried out on a sample (1 section in 6 for the whole of the length of the substantia nigra). For each section, the TH-positive neurones are counted separately on the two sides of the substantia nigra. The accumulated results for all the sections are expressed in the ratio: number of TH-positive neurones on the damaged side in relation to the number of TH-positive neurones on the undamaged side.

Behavioural Analysis

In order to evaluate the protective functional effects engendered by an injection of Ad-GDNF adenovirus on the lesion in the nigrostriatal tract, the sensorimotor performances of the animals are analysed during 2 behavioural tests: The test of the rotation induced by dopaminergic agonists (apomorphine, amphetamine and laevodopa), and the prehension (“paw-reaching”) test.

### Example 4

### Intrastriatal Injection of an Adenoviral Vector Expressing GDNF Prevents Dopaminergic Neuron Degeneration and Behavioral Impairment in a Rat model of Parkinson's Disease

An Ad-GDNF was constructed by inserting the coding sequence of the rat GDNF precursor protein (Lin et al., (1993) Science 260, 1130-1132) under the control of the LTR RSV promoter into a human type 5 E1E3 defective Ad (see above). A total of 1.5×10⁸ pfu of Ad-GDNF diluted in 9 μl PBS was injected into 9 sites (1 μl per site) of the striatum according to Horellou et al. ((1994) NeuroReport 6, 49-53) prior to lesioning with 6-OHDA. Control animals received either 1.5×10⁸ pfu of Ad-βGal virus diluted in 9 μl PBS or were naive animals that did not receive treatment before 6-OHDA. Also tested was the effect of sham-operation on amphetamine-induced turning after 6-OHDA by comparing the effect of intrastriatal injections of the vehicle (PBS) with naive animals that did not receive treatment before 6-OHDA.

To generate partial retrograde lesions, a rat model of Parkinson's disease described by Sauer and Oertel ((1994) Neuroscience 59, 401-415) was used. This lesion model was adapted to the virus injection procedure by injecting 6-hydroxydopamine (6-OHDA) in the center of the virus injection tracts in 3 deposits to obtain optimal protective effect of the virus. One or six days after injecting the virus, the rats were anaesthetized with equithesin (2 to 3 ml/kg, i.p.) and received a stereotaxic injection of 6-OHDA into their left striatum. Appropriate preparation of 6-OHDA is essential for the reproducibility of the lesion. 6-OHDA is unstable and its characteristics vary between batches. Therefore, one batch of 50 mg 6-OHDA-HCl (Sigma) was first divided into 4-5 mg aliquots and kept at −20° C. before use. To dissolve the toxin, a stock solution of ascorbate-saline (0.2 mg/ml, pH 4.30) was prepared on the day of the experiment and kept at 4° C. Each aliquot of 6-OHDA was dissolved immediately before use in ice-cold ascorbate-saline (6-OHDA-HCl, 4 μg/μl). The preparation was kept on ice and protected from light during the experiment. A total of 5 μl of 6-OHDA was infused at a speed of 1 μl/min and was equally distributed between three sites (the cannula was left in place another 4 min before being withdrawn) at the following coordinates: AP +1.2 mm from bregma; L +2.5 mm lateral to midline; V −5 mm, 4.6 mm and 4.2 mm ventral to dural surface (toothbar set at the level of the interaural line).

Histological Analysis. Following intrastriatal Ad delivery and 3 weeks after 6-OHDA injection, animals were perfused and their brains were processed for TH-immunohistochemistry as previously described by Horellou, et al. ((1994) NeuroReport 6, 49-53). The number of TH (+) cell bodies present in the substantia nigra (ventral tegmental area excluded) was determined in every sixth serial coronal section (14 μm thickness) between the coordinates AP −4.3 and −6.4 mm from bregma. A Zeiss microscope at high magnification (objective 20×) was used with the observer blind to the experimental group. DA survival was calculated as percentage of TH (+) cells counted in the contralateral non-lesioned SN. The degree of TH-innervation in the striatum was microscopically estimated by comparison with the density of TH (+) fibers observed on the contralateral non-lesioned side. Ten to 12 brain sections/animal (distributed between the coordinates AP +1.7 and +0.2 mm from bregma) were processed for TH-immunostaining. To assess general toxicity to the tissue of the various treatments, the size of the striatum was semi-quantitatively determined on the same TH-stained sections. The maximal lateral extension of the striatum was measured using an ocular microscope equipped with a grid (Zeiss) and compared with the contralateral non-lesioned striatum to calculate the percentage of atrophy.

To visualize in vivo βGal-transgene expression, 14 mm-thick coronal sections through the caudate putamen and the substantia nigra were processed for immunohistochemistry using specific polyclonal antibodies (Sabaté, et al., (1995) Nature Genet. 9, 256-260).

Behavioral Analysis. The injected animals were tested for amphetamine-induced turning 1, 2 and 3 weeks after intracerebral injection. Motor asymmetry was monitored in automated rotometer bowls (Imetronic, Bordeaux, France; (Ungerstedt, U. & Arbuthnott, G. W. (1970) Brain Res. 24, 485-493) for 90 min following an injection of D-amphetamine sulfate (Sigma, 5 mg/kg, i.p.). At the end of the session, the animals received a subcutaneous injection of 5 ml 5% glucose. A net rotation asymmetry score for each test was calculated by subtracting turns contralateral to the 6-OHDA lesion from turns ipsilateral to the lesion.

Statistical Analysis. All values are expressed as the mean+SEM. Differences among means were analyzed using one-factor analysis of variance (ANOVA). When ANOVA showed significant differences, pair-wise comparisons between means were tested by the Scheffé post-hoc test. Correlations were performed by calculating the correlation coefficient, and subsequent simple linear regression was performed. In all analyses the null hypothesis was rejected at the 0.05 level.

Results Demonstrating in vivo Protective Effect of Ad-GDNF

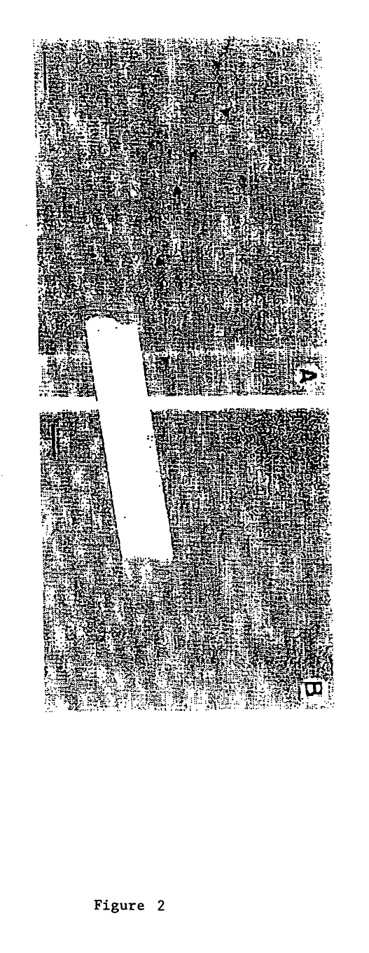

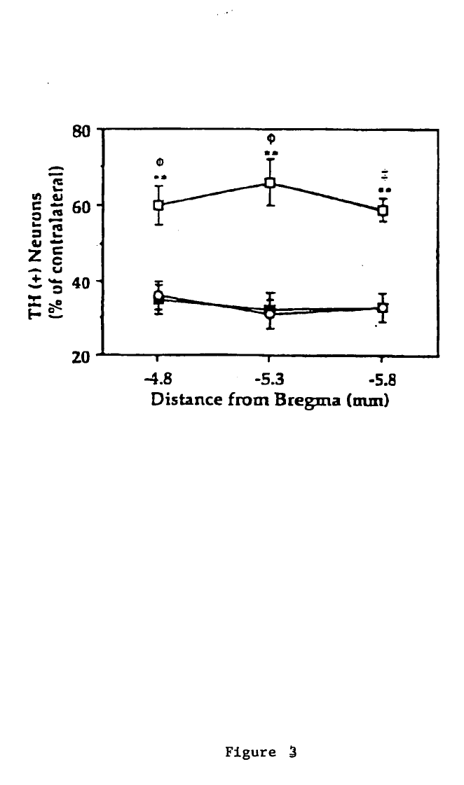

Correlation Between Dopaminergic-cell Survival in the Substantia Nigra and Turning Behavior: The efficacy of Ad-mediated GDNF gene transfer in vivo was tested in the rat model of Parkinson's disease of Sauer and Oertel which allows progressive degeneration of dopaminergic cells. GDNF was delivered at both dopaminergic-terminals and dopaminergic-cell bodies, by injecting the virus unilaterally into the striatum so as to obtain expression at the site of the injection as well as in the SN via retrograde transport of the virus. After 6 days, the rats received 6-OHDA in their previously injected striatum. This toxin injected into the striatum causes ipsilateral nigral dopaminergic-neuron loss (Sauer, H. & Oertel, W. H. (1994) Neuroscience 59, 401-415). Three weeks after the unilateral 6-OHDA lesion, the animals were sacrificed. Immunohistochemical analysis using specific anti-E. coli-βGal antibodies showed numerous infected cells in the injected striatum and in the ipsilateral substantia nigra (FIG. 2). Substantial transgenic βGal expression was detected for at least 4 weeks following adenoviral delivery. This suggests that Ad-GDNF drove a high level of production of transgenic GDNF. The survival of dopaminergic-neurons was analyzed throughout the substantia nigra between the coordinates AP −4.3 and −6.4 mm from bregma (FIG. 3 and Table 1). The animals treated with 6-OHDA alone or with Ad-βGal 6 days before the lesion showed a similar degree of dopaminergic-neuron degeneration. The survival of dopaminergic-cells was only about 30% throughout the substantia nigra. That for the Ad-GDNF group was 60-62%, showing a significantly better protection than in the 6-OHDA alone group (p=0.0003), or than in the Ad-βGal group (p=0.0009): twice as many dopaminergic-neurons survived in animals that received the Ad-GDNF than in those that did not or received Ad-βGal.

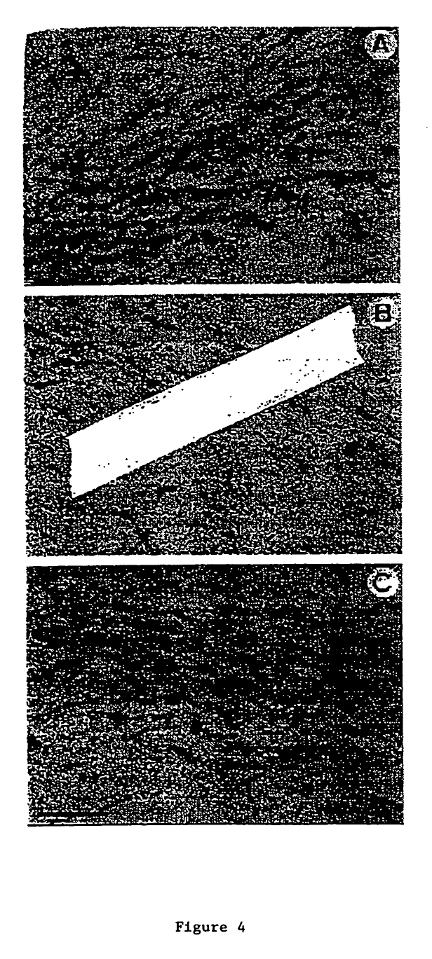

The Ad vector injected into the brain induces inflammation. Therefore the toxicity of the virus was investigated by histological analysis. The injected striatum of animals treated with Ad vectors were more inflammatory and atrophied than those treated with 6-OHDA alone (about 13% versus 2%, Table 1). The inflammation and atrophy induced by the Ad-βGal or the Ad-GDNF were not significantly different (Table 1). To evaluate the toxicity of the virus injection, we measured the number of dopaminergic-cells in animals that received Ad-βGal alone without injection of 6-OHDA. There was a reduction of 37±6% (n=4) in the number of dopaminergic-cells 3 weeks after intrastriatal injection (data not shown). Interestingly, adenoviral toxicity was not additive with 6-OHDA toxicity (Table 1). Ad-GDNF may compensate for not only the toxicity induced by 6-OHDA but also that induced by the first-generation Ad used in this study. The overall protective action of Ad-GDNF was not only apparent as an increased survival of dopaminergic-neurons but also as more tyrosine hydroxylase-innervation in the striatum and substantia nigra following 6-OHDA administration than in Ad-βGal-treated animals (Table 1, FIG. 4). Therefore, it appears that the Ad-GDNF injection in the striatum protected dopaminergic-cell bodies as well as dopaminergic-terminals in the striatum from the toxicity of 6-OHDA.

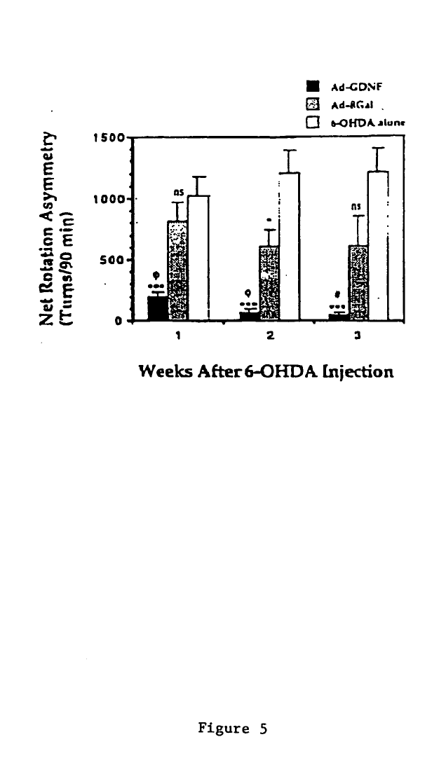

To evaluate the behavioral consequence of the dopaminergic-neuron degeneration, amphetamine-induced turning was monitored 1, 2 and 3 weeks following the lesion (FIG. 5). Control animals that received 6-OHDA had a mean rotation score of 1020+160 net ipsilateral turns over 90 min one week after the lesion. This turning behavior was stable for at least 3 weeks after the lesion. Injection of Ad-βGal 6 days prior to the lesion slightly decreased the rotation score as compared to 6-OHDA alone to 810±150 at 1 week post-lesion. The score decreased but not significantly, thereafter. The rotation score of the animals that received Ad-βGal was not significantly different from that of the animals that received 6-OHDA alone 1 week post-lesion (p=0.38). A statistical difference was observed 2 weeks post-lesion (p=0.03) but did not persist to the third week post-lesion (p=0.06) (FIG. 5). Injection of Ad-GDNF 6 days prior to the lesion reduced the rotation score to 200±30, 1 week post-lesion. The rotation score decreased further to 70±25 after 2 weeks and 47±17 after 3 weeks. The difference in rotation score between animals injected with Ad-GDNF and animals that received Ad-βGal (p=0.004, 0.002 and 0.03 at 1, 2 and 3 weeks, respectively) or 6-OHDA alone (p=0.0008, 0.0002 and 0.0001) was highly significant (FIG. 5).

We also tested the effect of sham-operation on amphetamine-induced turning after 6-OHDA. A group of animals were subjected to intrastriatal injections of the vehicle (PBS). Six days later, this group of animals and a group of naive animals received intrastriatal 6-OHDA and were tested for amphetamine-induced turning. The rates of rotation of these 2 groups were not significantly different (data not shown). Thus, neither the Ad-βGal injection nor the sham-operation induced a protective effect, demonstrating the functional effect of the Ad-GDNF in the model of Parkinson's disease used.

The correlation between the extent of dopaminergic-neuron survival in the substantia nigra and the rate of amphetamine-induced rotation 3 weeks after 6-OHDA injection was analyzed by plotting the two variables against each other. A significant correlation was found between amphetamine rotation (Y) and the percentage of surviving dopaminergic-cells (X): (Y=1652 −23.8X; r2=0.447; p=0.0003; n=25). As the groups of rats that received 6-OHDA alone or Ad-βGal before 6-OHDA had similar dopaminergic-cell survival rates (FIG. 3) and similar amphetamine-induced rotation rates (FIG. 5), they were pooled for this regression analysis (Y=1811 −27.5X; r2=0.259; p=0.03; n=18) than the other groups. Interestingly, the animals that received Ad-GDNF 6-days before 6-OHDA gave a regression curve with a much smaller slope (Y=187 −2.2X; r2=0.597; p=0.04; n=7). This difference in the linear regression curves illustrates the fact that animals that received the Ad-GDNF had a better motor functional score (lower amphetamine-induced rotation response) than predicted by their higher dopaminergic-cell survival rate in comparison to animals receiving 6-OHDA alone or 6-OHDA and Ad-βGal. Histological analysis showed a higher protection and/or sprouting of axonal terminal in the striatum and of dendrites in the substantia nigra of animals that received Ad-GDNF (Table 1 and FIG. 4). These observations suggest that not only the improved dopaminergic-cell survival but also the protection of dopaminergic-neurites in the striatum and/or in substantia nigra contribute to reduce turning behavior. It can be concluded that Ad-GDNF protected dopaminergic-cells in the substantia nigra and protected or stimulated dopaminergic-neurite arborisation resulting in better motor function.

Discussion

The capacity of Ad-mediated GDNF gene transfer to protect DA-neurons from the degeneration associated with Parkinson's disease has been evaluated. A rat model of the disease, obtained by intrastriatal injection of 6-OHDA which induces a progressive ipsilateral nigro-striatal degeneration, was used. Unlike lesions by intranigral injection of 6-OHDA, dopaminergic-nigral cells do not lose their dopaminergic phenotype but mostly undergo apoptosis (Sauer, H. & Oertel, W. H. (1994) Neuroscience 59, 401-415; Bowenkamp, ert al., (1996) Exp. Brain Res. 111, 1-7). Dopaminergic-cells start degenerating 1 week after 6-OHDA injection, but extensive death of nigral neurons is observed only 4 weeks post-lesion (Sauer, et al., (1995) Proc. Natl. Acad. Sci. USA 92, 8935-8939). In this paradigm, administration of recombinant GDNF protein to the substantia nigra, starting on the day of lesion, completely prevents dopaminergic cell death and atrophy (Sauer, et al. (1995)). Immunostaining using antibodies specific for E. coli-βGal protein shows the efficiency of the gene transfer using Ad vectors both in the striatum and in substantia nigra. In most animals, a large number of cells expressing the transgene was detected within the denervated striatum and in the substantia nigra. The labeled cells were dispersed throughout the entire caudate putamen with a pattern similar to that previously observed (Horellou, et al., (1994) NeuroReport 6, 49-53). The substantial βGal-transgene expression for at least 4 weeks following adenoviral delivery suggests that the protective effect observed following Ad-GDNF injection is probably due to the production of exogenous GDNF.

The sites of 6-OHDA injection were slightly less lateral and more dispersed (3 deposits along the needle track) than those described by Sauer and Oertel (1994). This led to a sustained amphetamine-induced rotation as early as 1 week after the lesion and for at least 3 weeks. Recently, Winkler et al. ((1996) J. Neurosci. 16, 7206-7215) reported that 2 deposits of 6-OHDA caused amphetamine-induced rotation. An important consequence of the simplicity of the behavioral test is that it facilitates the analysis of recovery following treatment. Amphetamine-induced rotation has been used previously as a marker of the dopaminergic depletion for partial lesions studies (Hefti, et al., (1980) Brain Res. 195, 123-137; Heikkila, et al., (1981) Brain Res. 195, 123-137). The dose used (5 mg/Kg) is a “standard rotational” (Björklund, et al., (1979) Brain Res. 177, 555-560) that has been widely used (Hudson, et al., (1993) Brain Res. 626, 167-174).

Amphetamine-induced rotation correlates significantly with DA-depletion in the lesioned striatum (Hudson et al., (1993)). In this work, the number of dopaminergic-cell bodies present in the substantia nigra of the lesioned animals was determined 3 weeks after 6-OHDA injection. A significant correlation between amphetamine-induced rotation and the extent of dopaminergic-cell loss in the lesioned substantia nigra following intrastriatal 6-OHDA lesion is shown. The comparison between animals receiving GDNF recombinant Ad and animals receiving the control Ad appears to be the most appropriate for determining the value of Ad as a tool to express GDNF. The inflammatory response, the destruction of neuronal tissue, and the gliosis induced in the striatum at the sites of injection were relatively limited and similar in both the Ad-GDNF and the Ad-βGal groups. A statistically significant difference in the rate of amphetamine-induced rotation and in the degree of dopaminergic-survival between the Ad-GDNF and the Ad-βGal groups was found. The protective effect of Ad-GDNF can be explained by expression of the transgene in the striatum and in the substantia nigra via retrograde transport of the Ad vector. Indeed, GDNF can be retrogradely transported by dopaminergic-neurons of the nigro-striatal pathway via a specific receptor-mediated uptake mechanism operating in the adult (Tomac, et al., (1995) Nature 373, 335-339). In this study, the availability of the neurotrophic factor to both the dopaminergic-cell bodies and to the dopaminergic-nerve terminals prevented not only dopaminergic-cell death but also striatal denervation. This most probably allowed the functional recovery that was observed following Ad-GDNF administration. This study suggests that GDNF expression in both striatum and substantia nigra not only allows protection of striatal dopaminergic innervating fibers and of dopaminergic-cell bodies but also limits motor impairment, suggesting possible therapeutic value of this method.

The Ad appears to have toxic effects: the size of the striatum was slightly reduced in both the Ad-GDNF and Ad-βGal injected animals; and 37% of dopaminergic-neurons were destroyed after Ad-βGal delivery (omitting toxin injection). The mechanism of this toxicity is not clearly understood. It may be caused by envelope virion particles, by expression of viral proteins and/or by antigen-mediated cytotoxicity (Byrnes, et al., (1996) J. Neurosci. 16, 3045-3055). It is thus likely that the Ad-GDNF compensates not only for the toxicity induced by 6-OHDA but also for that induced by the Ad. In this case, the protective activity of the Ad-GDNF may be higher than that observed. Increased therapeutic action may be observered using a third generation Ad-GDNF, a less toxic version recently developed (Yeh, et al., (1996) J. Virol. 70, 559-565), and by association with anti-inflammatory drugs to diminish the Ad-induced toxicity.

This work demonstrates that a recombinant Ad encoding GDNF significantly improved dopaminergic-cell survival in the substantia nigra and dopaminergic-neurite arborisation in substantia nigra and in striatum ipsilateral to the injection site. This effect is associated with reduction in turning behavior 1, 2 and 3 weeks following 6-OHDA lesion. These results suggest therapeutic value for Parkinson's disease using GDNF-gene transfer mediated by an adenoviral vector.

TABLE 1
Survival of DA neurons and degree of TH
innervation in 6-OHDA-lesioned rats subjected to
different treatments

| | | | Striatal | TH |
|---|---|---|---|---|
| Group | DA cells^{a} | TH SN^{b} | size^{c} | striatum^{d} |
| 6-OHDA (n = 10) | 31 ± 4 | + | −2.1 ± 0.7 | + |
| Ad-βGal (n = 8) | 31 ± 3^{†} | + | −13.6 ± 1.8* | + |
| Ad-GDNF (n = 7) | 62 ± 5‡ | +++ | −13.1 ± 2.3* | +++ |
| Animals were injected with 6-OHDA 6 days after treatment (n, number rats per group). Cornonal sections of SN and striatum were processed for TH immunohistochemistry. Values for DA cells and TH | | | | |
| # SN correspond to the analysis of five or six brain sections for each animal where TH⁺ cell bodies were counted only in the SN and restricted to the coordinate AP − 5.3 mm from bregma. Values for | | | | |
| # striatal size and TH strianum correspond to the analysis of 10-12 brain sections per animal (between the coordinates AP + 1.7 and +0.2 mm from bregma). DA cell bodies and DA neurites were more protected from 6-OHDA toxicity by Ad-GDNF than by Ad-βGal. | | | | |

*p < 0.001 versus 6-OHDA alone.
^{†}not significant versus 6-OHDA alone.
^{a}TH+ cells in SN (percent contralateral; mean ± SEM).
^{b}Estimation of TH-neurite density in SN: ++++, 100%; +++, 75%; ++, 50%; +, 25% contralateral.
^{c}Decrease in strianum size (percent contralateral; mean ± SEM).
^{d}Estimation of TH neurite density in the striatum (scale as above).

1. A replication defective recombinant adenovirus comprising at least one DNA sequence encoding the whole, or an active part, of GDNF or a derivative thereof.

2. An adenovirus according to claim 1, wherein the DNA sequence contains a secretory sequence in the 5′ position and in reading frame with the sequence encoding GDNF.

3. An adenovirus according to claim 1, wherein the DNA sequence is a cDNA sequence.

4. An adenovirus according to claim 1, wherein the DNA sequence is a gDNA sequence.

5. An adenovirus according to claim 1, wherein the DNA sequence encodes human GDNF.

6. An adenovirus according to claim 1, wherein the DNA sequence is under the control of signals enabling expression in nerve cells.

7. An adenovirus according to claim 6, wherein the expression signals comprise a viral promoter

8. An adenovirus according to claim 7, wherein the viral promoter is selected from the group consisting of E1A, MLP, CMV and RSV LTR promoters.

9. A replication defective recombinant adenovirus according to claim 1, comprising a cDNA sequence encoding human pre-GDNF under the control of the RSV LTR promoter.

10. A replication defective recombinant adenovirus according to claim 1, comprising a gDNA sequence encoding human pre-GDNF under the control of the RSV LTR promoter.

11. A replication defective recombinant adenovirus according to claim 1, comprising a DNA sequence encoding the whole, or an active part, of human glial cell-derived neurotrophic factor (hGDNF), or a derivative thereof, under the control of a tissue specific promoter enabling expression in nerve cells.

12. A replication defective recombinant adenovirus according to claim 11, wherein the promoter is the promoter of the neurone-specific enolase or the GFAP promoter.

13. An adenovirus according to claim 1, lacking regions of its genome which are necessary for its replication in a target cell.

14. An adenovirus according to claim 13, comprising the ITRs and a sequence enabling encapsidation, and in which the E1 gene and at least one of the genes E2, E4 and L1-L5 is non-functional.

15. An adenovirus according to claim 13, wherein said adenovirus is an Ad 2 or Ad 5 human adenovirus or a CAV-2 canine adenovirus.

16. A pharmaceutical composition comprising a replication defective recombinant adenoviruses according to claim 1.

17. A pharmaceutical composition according to claim 16, in an injectable form.

18. A pharmaceutical composition according to claim 16, comprising between 10⁴ and 10¹⁴ pfu/ml of defective recombinant adenoviruses.

19. A pharmaceutical composition according to claim 18, comprising between 10⁶ to 10¹⁰ pfu/ml of defective recombinant adenoviruses.

20. A mammalian cell infected with one or more replication defective recombinant adenoviruses according to claim 1.

21. A mammalian cell according to claim 20, wherein said cell is a human cell.

22. A mammalian cell according to claim 20, wherein said cell is a human fibroblast, myoblast, hepatocyte, endothelial cell, glial cell or keratinocyte.

23. An implant comprising infected cells according to claim 20, and an extracellular matrix.

24. An implant according to claim 23, wherein the extracellular matrix comprises a gel-forming compound.

25. An implant according to claim 24, wherein the gel-forming compound is selected from the group consisting of collagen, gelatin, glucoseaminoglycans, fibronectin and lectins.

26. An implant according to claim 23, wherein the extracellular matrix further comprises a support for anchoring infected cells.

27. An implant according to claim 26, wherein the support comprises polytetrafluoroethylene fibres.

28. A method of treating or preventing a neurodegenerative disease comprising administration to a patient suffering therefrom an effective amount of an adenovirus according to claim 1.

29. A method according to claim 28, wherein said disease is selected from the group consisting of Parkinson's disease, Alzheimer's disease, Huntington's disease, and ALS.


SEQUENCE LISTING (2 sequences)

SEQ ID NO: 1 - DNA (other nucleic acid), 36 bp; single-stranded, linear
  description: /desc = “Oligonucleotide”
ccgtcgacctaggccaccatgaagttatgggatgtc

SEQ ID NO: 2 - DNA (other nucleic acid), 35 bp; single-stranded, linear
  description: /desc = “Oligonucleotide”
ccgtcgacatgcatgagctcagatacatccacacc